(19) United States
(12) Patent Application Publication
(10) Pub. No.: US 2020/0283713 A1
(43) Pub. Date: Sep. 10, 2020

(54) METHODS AND SYSTEMS FOR CONTROL OF A FERMENTATION SYSTEM

(71) Applicant: Culture Biosciences, Inc., San Francisco, CA (US)

(72) Inventors: Matthew Adams Ball, Mountain View, CA (US); William Graham Patrick, San Francisco, CA (US)

(21) Appl. No.: 15/929,773

(22) Filed: May 21, 2020

Related U.S. Application Data
(63) Continuation-in-part of application No. PCT/US2018/061858, filed on Nov. 19, 2018.
(60) Provisional application No. 62/589,747, filed on Nov. 22, 2017.

(51) Int. Cl. C12M 3/00; C12M 1/26; C12M 1/36; C12M 1/34; G01N 35/00

(52) U.S. Cl. CPC C12M 23/44; C12M 33/00; C12M 41/48; G01N 35/0099; C12M 21/08; G01N 35/00871; C12M 41/36

(57) ABSTRACT

A fermentation automation workcell is provided. The fermentation automation workcell may permit automated seed train preparation, fermentation, and post-fermentation sample handling. The fermentation automation workcell may include one or more robotic handling devices that may aid in the automated process. A bioreactor array may include a plurality of modularized bioreactors that may be individually varied and/or controlled.

### CROSS REFERENCE

This application is a continuation-in-part of International Application No. PCT/US2018/061858, filed Nov. 19, 2018, which claims priority to U.S. Provisional Application No. 62/589,747, filed Nov. 22, 2017, each of which is incorporated herein by reference in their entirety.

### BACKGROUND

Fermentation processes have particularized requirements that need to be taken into account that require a controlled environment to conduct the bioreactions. Typical fermentation processes can be very time consuming and require a large amount of manual handling by a trained individual in a contaminant-free environment. For instance, seed preparation and sample prep and analysis may also require individualized attention in addition to the bioreactions. Such techniques can be particularly burdensome for an experiment that may benefit from high throughput fermentation.

### INCORPORATION BY REFERENCE

Each patent, publication, and non-patent literature cited in the application is hereby incorporated by reference in its entirety as if each was incorporated by reference individually.

### SUMMARY

A need exists for improved systems and methods for fermentation. A further need exists for providing high throughput, automated fermentation systems that allow for controlled variations in the fermentation process.

An aspect of the invention is directed to a system for automated fermentation, said system comprising: an automated seed train preparation station; a plurality of bioreactors configured to receive seed from the automated seed train preparation station, wherein at least one of the bioreactors of said plurality is removable and capable of having a different configuration from at least one other bioreactor of said plurality; and at least one robotic component configured to aid in automated seed train preparation at the automated seed train preparation station.

In some embodiments, the invention provides an apparatus for automated fermentation, said apparatus comprising: an automated seed train preparation station; a plurality of modular bioreactors configured to receive seed from the automated seed train preparation station, wherein at least one of the modular bioreactors of said plurality is removable and capable of having a different configuration from at least one other bioreactor of said plurality; and at least one robotic arm configured to aid in automated seed train preparation at the automated seed train preparation station.

In some embodiments, the invention provides a system for automated fermentation, said system comprising: an automated seed train preparation station comprising a fermentation agent; a plurality of modular bioreactors configured to receive seed from the automated seed train preparation station, wherein at least one of the modular bioreactors of said plurality is removable and capable of having a different configuration from at least one other bioreactor of said plurality; and at least one robotic arm configured to aid in automated seed train preparation at the automated seed train preparation station.

In some embodiments, the invention provides a method for automated fermentation, said method comprising: a system comprising: an automated seed train preparation station; a plurality of modular bioreactors configured to receive seed from the automated seed train preparation station, wherein at least one of the modular bioreactors of said plurality is removable and capable of having a different configuration from at least one other bioreactor of said plurality; and at least one robotic arm configured to aid in automated seed train preparation at the automated seed train preparation station.

Additional aspects and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of a fermentation automation workcell, in accordance with embodiments of the invention.

FIG. 2 shows an example of a fermentation automation workcell, in accordance with embodiments of the invention. 200: Automated fermentation workcell. 205: Sterile enclosure. 210: Seed train station. 211: Strain input. 212: Seed prep. 213: Shaker incubation. 214: Inoculation. 220: Fermentation station. 221: Bioreactor array. 222: Bioreactor. 223: Inset of a single bay. 224: Bay media bottles. 225: Single use reactor. 226: Magnetic agitation. 227: On-board camera. 228: Sampling location. 229: Pumps. 280: Control boards. 281: Heater/cooler. 230: Sample handling station. 231: Sample weighing. 232: Sample prep. 233: Sample analysis. 234: Sample output. 240: Bulk media bottles. 241: Scales. 242: Pumps. 250: Robot. 251: Robot carriage. 252: Linear rail. 253: End effector swapping. 260: Environment monitoring. 270: Camera.

FIG. 3 shows an example of a bay of a bioreactor array, in accordance with embodiments of the invention.

FIG. 4 shows examples of manifolds for bioreactors, in accordance with embodiments of the invention. 410: Bioreactor. 420: Inlet Gas. 430: Electricity. 440: Data. 450: Water. 460: Outlet Gas. 470: Off-gas analysis system.

FIG. 5 shows an example of a modular bioreactor array, in accordance with embodiments of the invention.

FIG. 6 shows an example of an electronics/communications architecture, in accordance with embodiments of the invention. 600: Electronics/Communications Architecture. 610: Cloud server. 620: Local area network. 630: Workcell computer. 631: Workcell embedded system. 632: Environment sensor embedded system. 633: Carriage pumping/scales embedded system. 634: Linear rail embedded system. 635: Sensors. 636: Scales. 637: Pumps. 638: Motor. 639: Sensors. 640: Robot. 650: Bay computer. 651: Bay embedded system. 652: Sensors. 653: Motor. 654: Pumping embedded system. 655: Pumps. 656: Scales. 657: Thermal embedded system. 658: Sensors. 659: Pumps. 659-1: Heater/Cooler. 660: Seed train computer. 661: Seed train embedded system. 662: Barcode reader. 663: Scales. 664: Incubator. 665: Incubator environmental sensor. 666: Live OD (optical density) sensing. 670: Sample analysis computer.

### DETAILED DESCRIPTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention provides systems and methods for fermentation. Various aspects of the invention described herein may be applied to any of the particular applications set forth below. The invention may be applied as a fermentation automation workcell, or an integrated system for data collection and analysis. It shall be understood that different aspects of the invention can be appreciated individually, collectively or in combination with each other.

Fermentation processes can be used for many applications. For instance, fermentation can be utilized for production of biomass (e.g., viable cellular material), production of extracellular metabolites (chemical compounds), production of intracellular components (e.g., enzymes and other proteins), or transformation of a substrate (e.g., the substrate itself may be a product). Fermentation processes are useful for biological experiments, drug manufacturing, food industry, biofuels, or many other applications. In some instances, it may be desirable to provide automated fermentation systems and methods that allow for low risk of contamination, high levels of accuracy and repeatability, high throughput, controlled variations, quicker turnaround, and/or require less manpower.

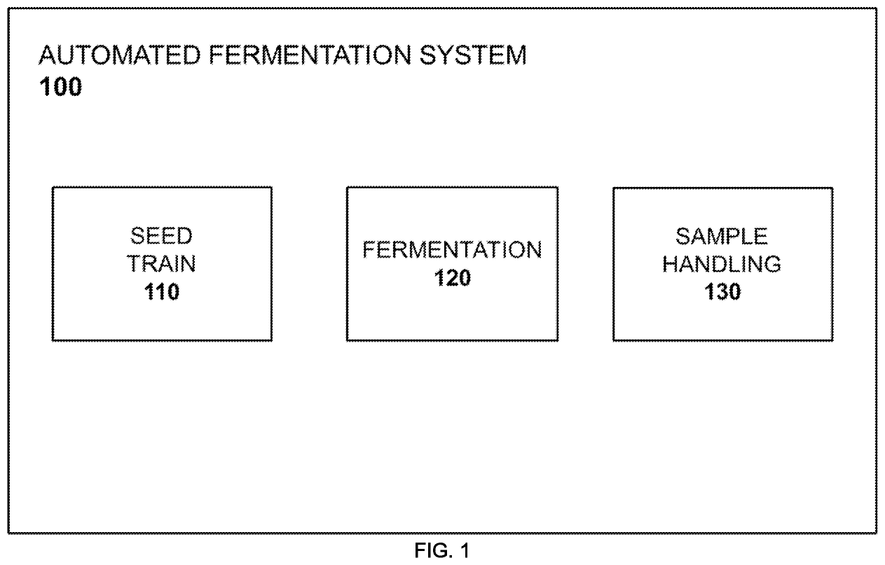

FIG. 1 shows a schematic of an automated fermentation system, in accordance with embodiments of the invention. An automated fermentation system 100 may comprise a seed train station 110, a fermentation system 120, and/or a sampling handling station 130. The automated fermentation system may optionally be a fermentation automation workcell, as described in greater detail elsewhere herein.

The automated fermentation system 100 may optionally include an enclosure. The automated fermentation system may comprise a sterile enclosure that prevents contaminants from coming inside the enclosure. The area within the automated fermentation system may be a clean, sterile environment, thereby reducing risk of contamination to the experiments. The enclosure may partially or completely enclose the seed train station, the fermentation station, and/or the sample handling station. The enclosure may or may not include a ceiling. The enclosure may or may not include, one, two, three, four or more walls. The enclosure may be substantially fluid-tight. The enclosure may be airtight and/or liquid-tight. The enclosure may or may not be capable of being taken down and put up repeatedly. In some embodiments, multiple enclosures may be provided. One or more stations or components may be provided in separate enclosures. Any description herein of an enclose may apply to an outer enclosure of the system. Any description herein may apply to one or more enclosures of the system.

The automated fermentation system may be of any size. For instance, the automated fermentation system may be the size of a facility, a room, a car, a benchtop, or may be a handheld or portable system. The enclosure may enclose the space of a facility, a room, a car, a benchtop, or may be a handheld or easily transportable item. In some instances, the system may be larger than, approximately the same size as, or smaller than a shipping container. One or more dimensions of the system (e.g., length, width, height, diagonal, diameter) may be less than or equal to 1 cm, 2 cm, 3 cm, 5 cm, 10 cm, 20 cm, 50 cm, 1 m, 1.5 m, 2 m, 3 m, 4 m, 5 m, 7 m, 10 m, 12 m, 15 m, 20 m, 25 m, 30 m, 35 m, 40 m, 50 m, 75 m, or 100 m. One or more dimensions of the system may be greater than any of the values provided, or fall within a range between any two of the values provided. The enclosure may have one or more dimensions less than any of the values provided. One or more dimensions of the enclosure may be greater than any of the values provided or fall within a range between any two of the values provided. In some embodiments, a maximum dimension of the system or enclosure (greatest of length, width, or height) may have a value less than any of the values provided, greater than any of the values provided, or falling within a range between any two of the values provided.

One or more processes within the automated fermentation system may be fully automated. One or more processes within the enclosure may be fully automated. A process may be automated and executed without requiring human intervention. A process may be automated when a human does not need to perform any manual manipulation. A process may be automated with aid of one or more processors. A process may be automated if the presence of a human is not required within an enclosure of the automated fermentation system. In some embodiments, seed train preparation 110, fermentation 120, and/or sample handling 130 may be fully automated. In some embodiments, transfer of materials from a seed train station to a fermentation station may be fully automated. In some embodiments, transfer of materials from a fermentation station to a sample handling station may be fully automated. In some embodiments, one or more preparation processes prior to fermentation may be automated. A fermentation process itself may be automated. Sample handling after fermentation may be automated. Sample handling may include sample preparation and/or analysis.

In some embodiments, one or more robotic components may aid in the automated processes. In some cases, one or more robotic components may comprise one or more robotic arms or other robotic components such as gantry. Any description herein of a robotic arm may apply to any type of robot or robotic component. For example, any description of an arm may apply to a gantry, such as a three-axis gantry. A robotic component may be capable of interacting with a seed train preparation station, a fermentation station, and/or a sample handling station. A robotic arm may aid in transfer of materials within a seed train preparation station, within a fermentation station, and/or within a sample handling station. A robotic arm may be capable of aiding in transfer of materials between a seed train preparation station and a fermentation station, or between a fermentation station and a sample handling station.

The automated fermentation system may provide an end-to-end, lights-off solution. In some embodiments, a strain or set of strains, and/or media may be an input to the system. In some embodiments, the input may be provided automatically. Alternatively, the input may be provided with human aid or intervention. An individual may manually provide the input. An output may comprise data and/or samples. The data may be collected with aid of one or more sensors and/or analytical equipment. The samples may be prepared, and may optionally be used for further analysis offline (e.g., outside the automated fermentation system). In some embodiments, the entirety of the processes between receiving the input and providing the output may be automated. The entirety of the process between the input and the output may occur without requiring human intervention or aid. The entirety of the process between the input and the output may occur without a human presence within the automated fermentation system or an enclosure thereof.

The automated fermentation system may comprise one or more bioreactors. In some embodiments, a fermentation station may comprise one or more bioreactors. Each bioreactor may correspond to an experiment. Each bioreactor may correspond to an individual fermentation process. Each bioreactor may be independently operating and/or controllable relative to another bioreactor. Any number or arrangement of bioreactors may be provided, as provided in greater detail elsewhere herein.

One or more modular components may be provided in the automated fermentation system. For instance, components, such as bioreactors and/or other equipment (e.g., analytical equipment, seed train preparation equipment) may be swapped in or out as needed. Modularity may be provided a seed train station, fermentation station, and/or sample handling station. In some embodiments, one or more robotic components may be modular. Modular functionality is described in greater detail elsewhere herein.

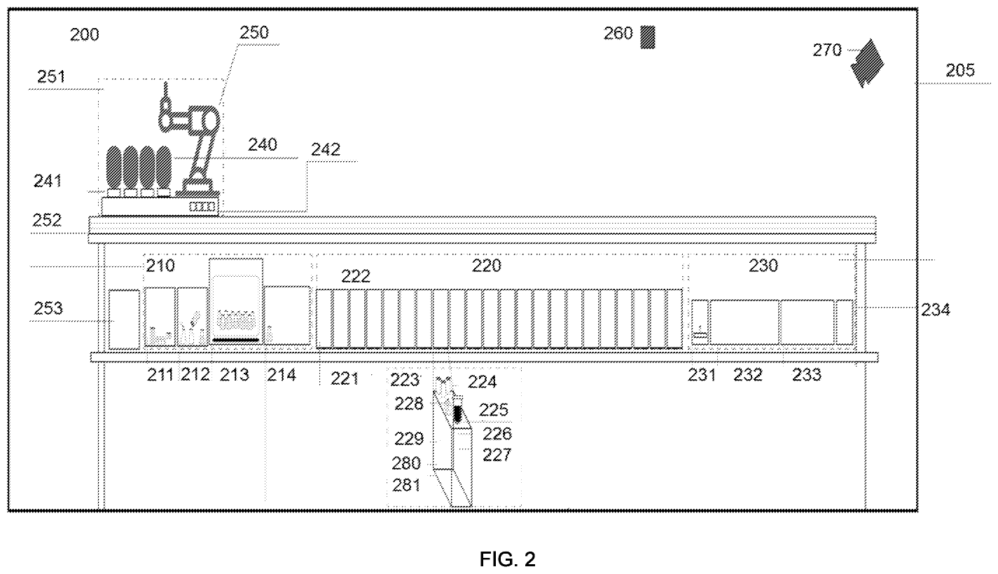

FIG. 2 shows an example of a fermentation automation workcell 200, in accordance with embodiments of the invention. A workcell may be an automated fermentation system. A workcell may comprise a sterile enclosure 205. A workcell and/or enclosure may have any qualities or characteristics of an automated fermentation system and/or enclosure as described elsewhere herein.

A workcell may comprise an automated seed train station 210, a fermentation station 220, and/or a sample handling station 230. Sample preparation and analysis is performed at the sample handling station 230. A workcell may also comprise one or more robotic components. Any description herein of a robot may apply to a robotic arm or other type of robotic component.

Any station described herein may or may not comprise a physical region within the workcell. A station may be spread out over multiple locations within a workcell. A station may be localized to a single location or region within a workcell. One or more components of a station may interact with one another. One or more components of a station may operate independently of one another. In some embodiments, one or more components of a station may operate in series, or in parallel.

A seed train station 210 may permit strain input 211, seed preparation 212, incubation 213, and/or inoculation 214. Optionally, a storage station, such as a cold storage station, may be provided, which may permit strains to be stored before they are used. Such activities may occur in the order provided or in any other order. Any of the processes may be optional or additional processes may be included. Any activities at a seed train station may be automated. In some embodiments, all activities at a seed train station may be automated. For instance, activities, such as strain input, seed preparation, shaker incubation, and/or inoculation may be performed automatically without requiring human intervention. One or more of the activities may be performed with aid of a robot.

Any activity at a seed train station may be monitored. For instance, seeds may be sampled as they are growing, and data about seeds may be collected. Data about seeds in the seed train station may be collected with aid of one or more sensors. The one or more sensors may or may not require the collection of one or more samples.

For strain input 211, a strain or set of strains may be provided to the workcell. Any description herein of providing a strain may be applied to providing a set of strains or multiple strains. A strain may be provided one or more containers. A strain may be provided via one or more tubes, frozen stocks, plates, beads, wells, channels, or any other technique. The strain may be provided manually or may be loaded in an automated fashion. In some embodiments, the workcell may be capable of accommodating multiples types of strain inputs. For example, the workcell may be able to accept one or more tubes, and one or more plates with a strain or set of strains. In some embodiments, a workcell may be automatically capable of identifying a type of strain input. For instance, the workcell may automatically identify a type of container that provides the strain. The workcell may automatically identify a strain material.

Media may be provided to the workcell. Media may be provided via one or more containers, such as bulk media bottles 240. The containers may be filled by a human operator and may be brought into the workcell. The filling may occur outside or within the workcell. The bottles may be brought into the bay. A robot may dispense the media into media bottles on the bay. A robot may dispense the media to a seed train station. Automated media preparation may occur.

In one example, during seed preparation 212, a robot may aid in filling containers, such as tubes or flasks, with media. In alternative embodiments, pipes, tubes, or channels may be employed to provide the media to tubes or flasks. A robot may add strains to the containers, such as the tubes or flasks. The robot may collect a strain from a container and transfer it to a tube or flask. In some embodiments, the media may be added first, and then the strains may be added. Alternatively, the strains may be added first, or media and the strains may be added concurrently. The media and strains may be provided to different containers. In some instances, the media containers will be transferred to the bioreactors, while the strains may undergo further processing. A robot may aid in transfer of a material, such as media or a strain by grasping and lifting an object and/or pouring the material. The robot may aid in transfer of a material by pipetting the material from one container to another. The robot may take any other action in transferring the material.

Optionally, one or more sensors may be provided. In some embodiments, a sensor may be used to track initial media volume. One or more sensors may be employed to track strain volume or type. One or more sensors may be employed to determine any material quantity (e.g., volume, weight, height, density, concentration, or other measurement).

During incubation 213, containers may be added to an incubator. Optionally, incubation may or may not include shaking. Any description herein of shaker incubation may apply to any type of incubation which may or may not include a shaker. A robot may aid in one or more activities during shaker incubation. For instance, a robot may open a shaker/incubator. A robot may add containers, such as flasks or tubes to the shaker/incubator. The containers may contain strain and/or media. Robot may optionally close a shaker/incubator after the containers have been added. A robot may open or close a shaker/incubator with aid of a gripper, magnets, suction, or any other technique. Any number of shakers/incubators may be provided. For instance, a single shaker/incubator, two shakers/incubators, three shakers/incubators, four shakers/incubators, five shakers/incubators, or move may be provided. Each shaker/incubator may be capable of operating independently of one another. Each shaker/incubator may have independent settings that can be adjusted.

In some instances, one or more sensors may be provided. A sensor may provide live optical density (OD) monitoring of each culture. A temperature sensor may be provided within a shaker/incubator. An open close sensor may detect when a shaker/incubator is open/closed. This may be useful for determining whether a door is properly closed when it should be, or when a robot needs to open or close a door. In some embodiments, data from one or more sensors may be used to affect operation of a shaker/incubator. For example, data from an OD sensor and/or temperature sensors may be used as feedback for operation of a shaker/incubator. Alternatively, data from the sensors may not affect operation of the shaker/incubator. For example, optical density may be measured as a data point. Data from one or more sensors may affect operation of a robot. For instance, a robot may be instructed to interact with the shaker/incubator or containers within the shaker/incubator based on data from one or more sensors.

During inoculation 214, a robot may transfer an inoculation volume from a container to another container. For example, a robot may transfer an inoculation volume from a flask into a tube. A robot may transfer an inoculation volume from a container that was within the shaker/incubator to a container that will be transferred to a bioreactor. The robot may transfer the inoculation volume using any technique. For example, the robot may pipette the inoculation volume from the first container to the second container. Optionally, the robot may pick up and pour selected volume of the first container into the second container. The inoculation volume may comprise the materials have that undergone shaker incubation. The inoculation volume may comprise strain that has undergone the shaker incubation.

The inoculation volume may be transferred to a bioreactor at a fermentation station. For instance, the second container, such as a tube or any other type of container, may be transferred to a bioreactor. The second container may be a single-use vessel. In some embodiments, the container used at the bioreactor may be disposable. Alternatively, the container may be reusable.

One or more optional sensors may be provided for use during inoculation. For instance, a quantity of the inoculation volume may be measured (e.g., volume, weight, height, density, concentration, or other measurement). For instance, a scale may be employed to measure the inoculation volume. Optionally one or more optical devices may be provided. For example, a barcode reader may be employed to recognize one or more barcodes (e.g., 1D code, 2D code, 3D code, QR code, etc.). An optical device, or scanner, may be capable of reading and recognizing any visual marker. This may aid in identification of the inoculation volume and tracking the presence and/or location of the inoculation volume within the workcell.

A fermentation station 220 may comprise a bioreactor array 221. A bioreactor array may comprise one or more bioreactors 222.

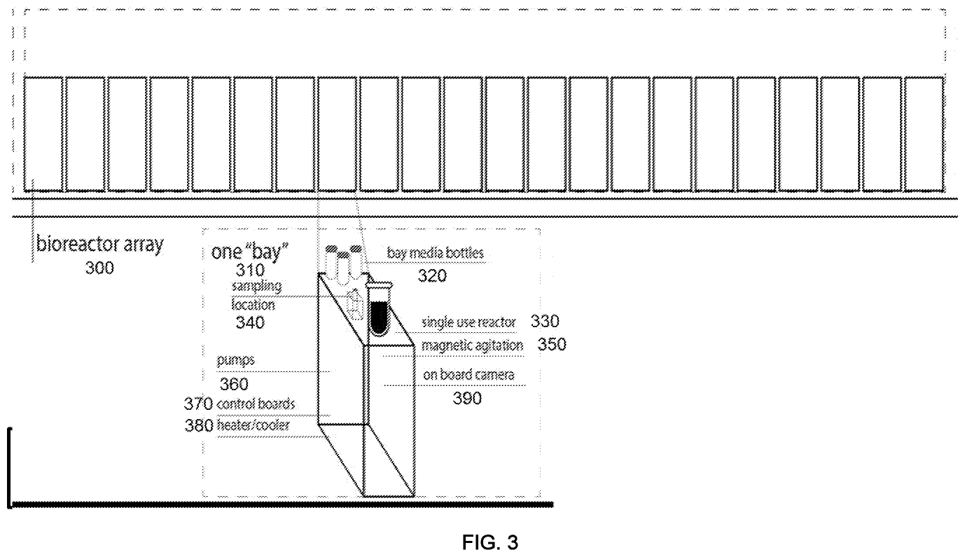

FIG. 3 shows an example of a bay of a bioreactor array 300, in accordance with embodiments of the invention. The bioreactor array may be provided at a fermentation station of a workcell, as discussed elsewhere herein. A bioreactor array may comprise one or more bioreactors 310. The bioreactors may also be referred to as bioreactor bays (or bay), reactor vessels, or bioreactor modules. A fermentation station may comprise a plurality of bioreactors. The bioreactors may be arranged in any fashion. A bioreactor array may comprise a single row of bioreactors, multiple rows of bioreactors, a single column of bioreactors, multiple columns of bioreactors, a single stack of bioreactors, or multiple stacks of bioreactors. A bioreactor array may be an m×n array of bioreactors, or an mxnxp array of bioreactors, where m, n, and p are whole numbers of 1 or greater. Optionally, m, n, orp may be greater than or equal to 1, 2, 3, 4, 5, 6, 7, 8, 9, 10, 12, 15, 20, 30, 40, 50, or 100. Optionally, m, n, or p may be less than any of the number provided or fall within a range between any two of the numbers provided.

Any number of bioreactors may be provided within a bioreactor array. For instance, a bioreactor array may comprise 1 or more, 2 or more, 4 or more, 6 or more, eight or more, 10 or more, 12 or more, 18 or more, 24 or more, 36 or more, 48 or more, 60 or more, 96 or more, 128 or more, 256 or more, or any other number of bioreactors. The bioreactor array may comprise less than any of the numbers provided herein, or fall within a range between any two of the numbers provided herein.

Any number of bioreactor arrays may be provided at a fermentation station. For instance, a single bioreactor array may be provided at a fermentation station. Alternatively, a plurality of bioreactor arrays (e.g., two or more, three or more, four or more, five or more) bioreactor arrays may be provided at a fermentation station.

In some embodiments, each bioreactor within a bioreactor array may be capable of operating independently of other bioreactors within the bioreactor array. Each bioreactor array may be capable of operating independently of other bioreactor arrays.

A single robot may serve a single bioreactor. Alternatively, a single robot may serve multiple bioreactors. In some instances, a single robot may serve at least 1, 2, 4, 6, 8, 12, 18, 24, 36, 48, 96, 128, 256, or more bioreactors. Alternatively, a single robot may serve fewer bioreactors than any of the values listed herein, or a number of bioreactors falling within a range between any two of the values provided herein. In some embodiments, a single robot may serve a single bioreactor array. Optionally, multiple robots may serve a single bioreactor array. A single robot may serve multiple bioreactor arrays. In some instances, each bioreactor may have one or more dedicated robots. Optionally, multiple robots may be provided that may each serve multiple bioreactor arrays.

A bioreactor may have any dimension. For instance, a bioreactor may have a dimension (e.g., length, width, height, diagonal, or diameter) less than or equal to 1 cm, 3 cm, 5 cm, 10 cm, 15 cm, 20 cm, 25 cm, 30 cm, 35 cm, 40 cm, 45 cm, 50 cm, 60 cm, 70 cm, 80 cm, 90 cm, 1 m, 1.2 m, 1.5 m, or 2 m. A bioreactor may have a maximum dimension less than or equal to any of the values provided herein. A bioreactor may have a dimension greater than any of the values provided herein or falling within a range between any two of the values provided herein. A bioreactor may have a maximum dimension greater than any of the values provided herein, or falling within a range between any two of the values provided herein.

Optionally, a bioreactor may be a single-use bioreactor. The bioreactor may be disposable. Alternatively, the bioreactor can be reused. In some embodiments, one or more components of a bioreactor may be single-use/disposable. One or more components of a bioreactor may be reusable. A bioreactor may be removable from fermentation station. A bioreactor may be attached, detached, and/or reattached at the fermentation station. A bioreactor may be exchangeable with another bioreactor. In some embodiments, one or more receiving interfaces may be provided at a fermentation station, which may each be capable of receiving a bioreactor. In some embodiments, each receiving interface may be identical. A bioreactor may fit into any of the receiving interfaces at a fermentation station. Various bioreactors may be received at a receiving interface. In some instances, bioreactors with different settings or different configurations may be received at a receiving interface.

Each bioreactor (or ‘bay’) 310 may comprise one or more of the following: media container 320, reactor vessel 330, sampling location 340, agitator 350, pump 360, control board 370, heater/cooler 380, camera 390, a condenser for exhaust gas, and/or a location for ‘run-time’ media additions.

A media container 320 may optionally be received from a seed train station of a workcell. The media container may comprise one or more media bottles. In another embodiment, a media container may be provided at a bioreactor and media may be provided to the media container with aid of a robot. The media container may be removable or detachable from the bioreactor. The media container may be single-use or reusable. The bioreactor may have a media container receiving region which may support and hold one or more media containers.

A reactor vessel 330 may be provided at a bioreactor. The reactor vessel may optionally be received from a seed train station of a workcell. The reactor vessel may comprise one or more containers, such as tubes, flasks, wells, plates, or any other type of container as described elsewhere herein. In another embodiment, a reactor vessel may be provided at a bioreactor and seed that has undergone seed train preparation may be added to the reactor vessel with aid of a robot. The reactor vessel may be removable or detachable from the bioreactor. The reactor vessel may be single-use or reusable. The bioreactor may have a reactor vessel receiving region which may support and hold one or more reactor vessels. A bioreaction may occur within the reactor vessel. For instance, a fermentation process may occur within a reactor vessel while coupled to the bioreactor. A reactor vessel may be formed from any material. For example, the reactor vessel may be formed from injection molded plastic, wood, iron, copper, glass, stainless steel, or other materials. The reactor vessel may be formed from a material that is substantively not corrosive, may be capable of tolerating high pressure, may be able to resist pH changes, may be able to tolerate steam sterilization, and/or may be free of toxins. Any description herein of experiments that may be conducted within a workcell may include a fermentation process that may occur in a bioreactor (e.g., reactor vessel of a bioreactor).

A sampling location 340 may be provided at a bioreactor. One or more sampling containers may be provided to the sampling location. In some embodiments, a sample within a sampling container may be collected from a reactor vessel 330. Sampling may occur at a single point during an experiment, or multiple points during an experiment. For example, multiple samples may be collected over time during an experiment. A separate sampling container may be used for each collection. In some embodiments, media may optionally be added from a media container to a reactor vessel. Media may be added from a media container to a reactor vessel at any point at the beginning of an experiment. The media may be added to a media container at a single point in time, or multiple points in time during an experiment. Media may or may not be added directly to a sampling container. In some embodiments, a sampling container may be stored at a sampling location until the sampling container is picked up and/or transported to a sampling handling station 230. Sampling containers at a sampling location may or may not undergo further fermentation.

In some embodiments, an agitator 350 may be provided at a bioreactor. For example, an agitator may provide magnetic agitation. In some instances, mechanical agitation, such as blades, may be employed. One or more impellers and/or baffles may be employed to aid in agitation. One or more agitation components (e.g., impeller) may be formed using 3D printing techniques. Agitation may be provided to contents of a reactor vessel. In some instances, agitation may be provided continuously. The level of agitation may be constant or may vary over time. In some instances, agitation may only be provided at selected time periods.

A bioreactor may comprise one or more pumps 360. The one or more pumps may control flow of a fluid, such as a liquid feed. A pump may remove liquid from the reactor vessel or to add acids and bases, antifoam reagents, and nutrients for continuous or batch cultures. The one or more pumps may control a flow of gas. For example, air or other gases may be added to the reactor vessel. In some embodiments, peristaltic pumps may be employed.

A bioreactor may have a control board 370. The control board may comprise one or more processors that may execute code, logic or instructions to perform one or more steps. The control board may generate instructions that may affect operation of the agitator, the pumps, heater/cooler, camera, sensors, and/or material handling. A control board may optionally control flow of one or more fluids. For instance, a control board may control flow of one or more gases into or out of a reactor vessel. A control board may control flow of one or more materials, such as media, to or from the reactor vessel.

The control board may generate instructions that may affect operation of one or more components of the bioreactor. The control board may generate instructions that may affect operation outside the bioreactor. The control board may receive instructions from other sources. For instance, the control board may receive instructions from other bioreactor control boards, from the cloud, from the robots, from any components within the system, or any components outside the system. For example, the instructions may be conveyed to a robot that may interact with the bioreactor. A bioreactor may comprise one or more memory storage units. The one or more memory storage units may comprise non-transitory computer readable media that may comprise code, logic, or instructions for executing one or more steps. The control board may execute one or more experiment protocols. A memory storage unit may store instructions for a particular experiment for the bioreactor.

A temperature control system 380 may be provided for a bioreactor. The temperature control system may comprise a heater and/or cooler that may control the temperature of the bioreactor. The temperature control system may control the temperature of the contents of a reactor vessel. In some embodiments, the temperature control system may be able to provide temperature control to the precision of at least 0.01 degrees C., 0.05 degrees C., 0.1 degrees C., 0.5 degrees C., 1 degree C., 2 degrees C., 3 degrees C., or 5 degrees C. Optionally, a temperature control system may comprise a water bath. A water bath may cool or heat a reactor vessel. In some instances, a temperature control system may comprise thermoelectric heating components. In some instances, Pelletier devices may be used.

A bioreactor may have an on-board camera 390. The on-board camera may be able to visualize the reaction taking place. For instance, the on-board camera may be able to capture images of the contents of the reactor vessel. In some instances, one or more on-board cameras may capture images of the sampling location and/or media containers. An on-board camera may be useful for detecting a stage of an experiment, and/or positioning of any physical components of the bioreactor.

A bioreactor may optionally have a condenser for exhaust gas. The condenser may be provided for any type of gas that may be generated within the bioreactor. The condenser may be contained partially or completely within the bioreactor, or supported by the bioreactor.

In some instances, a bioreactor may comprise a location for ‘run-time’ media additions. In some instances, a media bottle may be provided that may be used for one-time media additions in the middle of an experiment. Any number of containers may be provided that may add materials, such as media, at any point during an experiment, which may occur on the bioreactor. The media additions may be partially or completely within the bioreactor, or supported by the bioreactor.

A bioreactor may comprise a housing or a substrate that may support one or more components of the bioreactor. A housing may partially or completely enclose one or more components of the bioreactor. In some instances, a bioreactor may comprise a head plate. Optionally, a bioreactor, housing, substrate, or head plate may be formed using 3D printing techniques.

A bioreactor may or may not have a local power source. For instance, a bioreactor may have an on-board energy storage system, such as a battery or capacitor. In some instances, a bioreactor need not have an on-board energy storage system and may receive power from another part of the workcell. In some instances, a fermentation station receiving interface may provide power to a bioreactor.

A robot may interact with a fermentation station 220. A robot may interact with one or more bioreactors 222. The bioreactors may have any of the qualities or characteristics described for FIG. 3. A robot may move one or more containers of the bioreactor. For instance a robot may move a sampling container to or from a sampling location. A robot may also move a sampling container from a sampling location to a sample handling station. A robot may interact with media containers. For instance, a robot may add or remove caps or other closures from the media containers. The robot may dispense liquids to or from the media bottles. In one example, a robot may dispense a media to a bioreactor media container from a seed train station 210 or from bulk media bottles 240. A robot may optionally dispense media from a bioreactor media container to a reactor vessel or other component of the bioreactor. Alternatively, the media may be automatically dispensed to a reactor vessel or other component of the bioreactor with aid of built-in tubing, piping, channels, or other techniques. In some instances, a robot may load a vessel into a bay. The robot may make any necessary liquid or fluid connections required. For example, a robot may put pumping lines into a peristaltic pump. The robot may also unload the vessel. The robot may make any necessary liquid or fluid disconnections when unloading the vessel. The robot may optionally put an unloaded vessel into a waste area. A robot may also optionally add liquids or other materials to a post-sterile addition system. A robot may add liquids to the bioreactor during an experiment. These may be referred to as runtime additions or post-sterile additions. Optionally, they are not continuous or semi-continuous feeds. Instead, they may happen once. For example, a robot may add anti-foam in response to foaming. A robot may add a certain molecule which may induce product formation. The robot may just add a certain media component that may not be needed at the beginning of an experiment but may be needed later on.

Each bioreactor may comprise one or more sensors. For example, one or more of the following sensors may be provided: temperature sensor, dissolved oxygen sensor, pH sensor, biomass concentration sensor (e.g., may measure optical density or other characteristics), UV Vis/Raman sensor, scales, and/or camera may be provided. Sensors may be reusable or may be single-use sensors. The sensors may be able to measure a quality of one or more components of the bioreactor, such as a reactor vessel, sampling location, media container, or any other component of the bioreactor. In one example, a bioreactor camera may be employed to visualize the contents of a reactor vessel or sampling container (e.g., color, tracking foam, tracking volume levels, etc.). The sensors of each bioreactor may be capable of operating independently of sensors on-board other bioreactors.

A sensor can be used to determine the foam level, emitted infrared light, emitted UV light, and emitted visible light from a bioreactor array. A sensor or device that is a part of a system or apparatus disclosed herein can measure for example, the mass inflow and volumetric outflow of air, nitrogen, oxygen, carbon dioxide, and methane.

A sample handling station 230 may permit sample preparation and/or analysis. A sample handling station may comprise one or more components for sample weighing 231, sample preparation 232, sample analysis 233, sample storage, and/or sample output. Any of these components or steps may be optional, provided in any order, or additional components or steps may be provided.

One or more samples may be weighed 231. The sample may be weighed within a sample container. The sample container may be provided from a fermentation station 220 or from the seed station. The sample may be provided from one or more bioreactors of a fermentation station. The sample may be provided within a container that was used at one or more bioreactors. The sample may be collected from a container that was used at one or more bioreactors and provided to a new container that is used for sample weighing. The sample and/or sample container may be provided with aid of a robot. The sample may be provided during any stage of experimentation. For instance, the sample may be provided to the sample handling station (e.g., for sample weighing) after completion of an experiment at a bioreactor. In some instances, the sample may be provided at the beginning, or any point during an experiment at the bioreactor. Multiple samples may be provided at multiple points in time.

Sample weighing may occur with aid of one or more sensors. For example, a scale may be employed to weigh the sample. The scale may have a high degree of accuracy and/or precision. In some instances, the scale may at least be accurate on the order of 0.00001, g, 0.00005 g, 0.0001 g, 0.0005 g, 0.001 g, 0.005 g, 0.01 g, 0.05 g, 0.1 g, 0.2 g, 0.5 g, 1 g, 2 g, 3 g, 5 g, 10 g, 15 g, 20 g, 30 g, or 50 g. Other techniques may be employed to detect an amount of sample (e.g., weight, volume, concentration, density, etc.).

An optical sensor, such as a barcode reader may also be employed to aid in sample measurement. For example, the optical sensor may read a symbol, such as a barcode, to detect the sample information, container information, source of the sample, experiment information relating to the sample, or any other information. The symbol may be used to track the sample, and information about the experiments conducted on the sample, the bioreactor used for the sample, or any other data of the sample may be added and/or accessible.

Sample preparation 232 may occur after sample weighing, concurrently with sample weighing, or subsequent to sample weighing. In some instances, sample preparation may occur with aid of one or more robots. Human intervention may not be required for sample preparation. A robot may operate equipment and perform liquid handling. The robot may be capable of interacting with and/or operating off-the-shelf equipment that does not requirement any modification to be used to by the robot. The robot may manipulate one or more sets of controls for the equipment (e.g., pressing buttons, flipping switches, turning dials, touching a touchscreen, opening/closing doors, etc.).

In some instances, sample preparation may comprise centrifugation. One or more centrifuges may be provided. A single centrifuge may accommodate a single sample at a time or multiple samples at a time. When multiple centrifuges are provided, they may be capable of operating independently of one another.

Sample preparation may include one or more separation processes. For instance, separation of cell pellet from supernatant may occur. Centrifugation may aid in separation, or other techniques or equipment may be used for separation.

Optionally, sample preparation may comprise additions of materials to the sample. For instance, liquid additions may be provided to lyse/stabilize cells or stabilize some analyte. Any type of materials may be added for sample lysing, stabilization, marking, reactions, or any other desired effect.

One or more sensors may be provided which may monitor activities of the various equipment and/or sample status. Optionally, data from the sensors may be used as feedback that may affect sample preparation.

Sample analysis 233 may occur after sample preparation, concurrently with sample preparation, or subsequent to sample preparation. In some instances, sample analysis may occur with aid of one or more robots. Human intervention may not be required for sample analysis. A robot may operate equipment for analysis. The robot may be capable of interacting with and/or operating off-the-shelf equipment that does not requirement any modification to be used to by the robot. The robot may manipulate one or more sets of controls for the equipment (e.g., pressing buttons, flipping switches, turning dials, touching a touchscreen, opening/closing doors, etc.). The robot may comprise a camera that may allow the robot to visually detect equipment, samples or other components of the system. For instance, a camera on the robot may allow the robot to read a screen or recognize controls of equipment, and may facilitate robot interaction with equipment. In some instances, the equipment for analysis may interface with equipment for sample handling. Alternatively, the equipment for analysis may operate independently of equipment for sample handling.

Sample analysis may optionally occur without requiring the aid of one or more robots. In some instances, equipment may be controlled without requiring robotic interaction. Additional methods to digitally control equipment may be employed. The workcell may communicate with the equipment to provide instructions for control or to read data collected by the equipment. In some instances, a device separate or external to the workcell (e.g., via the cloud) may communicate with the equipment to provide instructions for control or to read data collected by the equipment.

Equipment used for sample analysis may include, and is not limited to, equipment for biochemical analysis, ultraviolet (UV)/visible (Vis)/infrared (IR), high-performance liquid chromatography (HPLC)/gas chromatography (GC)/mass spectrometry (MS), Raman spectroscopy, DNA sequencing, RNA sequencing, protein quantification, cell-counting, cell imaging, microscope, or any other type of equipment. The sample may be analyzed for composition, properties, emissions, quantity, density, concentration, or any other quality or characteristic.

Data from the sample analysis may be further analyzed within the workcell or outside the workcell. Optionally, data generated from the sample analysis may be used as a control signal for bioreactor control.

In some instances, sample storage may be provided. Samples may be stored in a workcell for any length of time. For instance, samples may be stored in a manner that they may remain stable for at least 1 minute, 5 minutes, 10 minutes, 20 minutes, 30 minutes, 45 minutes, 1 hour, 2 hours, 3 hours, 4 hours, 6 hours, 12 hours, 24 hours, 36 hours, 48 hours, 72 hours, or longer. In some instances, the samples may be stored in cold storage. For instance, the samples may be stored in a cold container that may keep the samples below a desired temperature threshold.

Optionally, sample output 234 may be provided. Physical sample may be stored or provided outside the workcell. The physical sample storage may occur automatically without requiring human intervention. The sample may be provided in a manner that may allow the sample to be collected outside the workcell. In one example, cold storage of sample may be provided. A robot may aid in putting samples into cold storage. Samples may be broth or prepped in some manner (e.g., centrifugation or any other sample preparation step). In some instances, robots may aid in putting samples into desirable storage conditions (e.g., controlled temperature, controlled exposure to light or other radiation) and/or desired storage locations. A robot may put samples into liquid nitrogen to flash freeze them. A robot may aid in putting sample into a rack or box that a technician may be able to pick up. Optionally further handling or analysis of the sample output may occur outside the workcell.

In some embodiments, a workcell may permit cleaning up at the end of one or more experiments. This may include the removal of single use vessels. For example, the bioreactor vessels may be removed from the bioreactors when the experiment is concluded. The cleanup may occur automatically with aid of one or more robots. The cleanup may occur without requiring human aid or intervention. A robot may be capable of picking up vessels or other containers that are no longer needed and moving them to a different location. The removed containers may be sterilized, washed, or cleaned, for reuse. In some instances, this step may occur automatically without requiring human intervention. In some instances, the removed containers may be disposed or removed from the workcell.

One or more bulk media containers 240 may be provided within a workcell. The bulk media containers may comprise any type or number of containers (e.g., bottles, flasks, tubes, plates, wells, etc.). The bulk media containers may be entirely enclosed from the environment. Alternatively, one or more openings may be provided that may allow for exposure to the environment.

A bulk media container may be filled by a human operator. The containers may be filled inside or outside the workcell. Optionally, a robot may dispense media into the bulk media containers. A robot may or may not directly handle the bulk media container. In some instances, robots may be employed to handle media from the bulk media container and provide it to other containers within the workcell. In some embodiments, media may be directly metered and/or fed to other containers within the workcell. Media may be directly fed to one or more containers within a bioreactor.

Media containers may optionally be provided on scales 241 which may allow for precise metering of media. One or more pumps 242 may aid in dispensing the media. The pumps may dispense media from the bulk media containers. Alternatively or in addition, pumps may be employed to dispense media to the bulk media containers. In one example, pumps may be used to dispense media from the bulk media containers to one or more container at a seed train station 210, a fermentation station 220, and/or a sample handling station 230. Alternatively or in addition, robots may be employed to provide media from the bulk media containers to one or more containers at a seed train station 210, a fermentation station 220, and/or a sample handling station 230.

A workcell may comprise one or more robots 250. Any description here of a robot may apply to a robot arm, and vice versa. Any description of a robot may comprise one or more robotic components capable of actuation. A robot may comprise a robot arm. The robot arm may be a 6-axis robot arm. The robot arm may be capable of motion about 1 or more, two or more, three or more, four or more, five or more, or six or more axes of motion. The robot arm may comprise one or more, two or more, three or more, four or more, five or more, six or more, seven or more, eight or more, nine or more, or ten or more joints. The joints may comprise motors that may allow various support members to move relative to one another. The robot arm may comprise one or more, two or more, three or more, four or more, five or more, six or more, seven or more, eight or more, nine or more, or ten or more support members. In one example, a first support member may bear weight of an end effector. A second support member may bear weight of the first support member and/or the end effector, and so forth. The motors may allow rotation of one or more support members relative to one another. One or more sliding mechanism may be provided that may allow lateral displacement. One or more telescoping components for support members may or may not be provided. The robot arm may have a free range of motion that may match or exceed the range of motion of a human arm. Ball and socket joints may or may not be employed by the robot arm. A robot arm can move an inoculant from an automated seed system to the reactor.

The robot may have any dimensions. In some instances, a dimension (e.g., length, width, height, diagonal, diameter) may be at least 1 cm, 3 cm, 5 cm, 7 cm, 10 cm, 15 cm, 20 cm, 30 cm, 40 cm, 50 cm, 60 cm, 70 cm, 80 cm, 90 cm, 1 m, 1.2 cm, 1.5 m, 1.7 m, 2 m, 2.5 m, or 3 m. The dimension may be less than any of the values provided or may fall within a range between any two of the values provided. The robot may have a maximum dimension that is less than any of the values provided herein, greater than any of the values provided herein, or falling within a range between any two of the values provided herein.

The robot may comprise a robot carriage 251. In some embodiments, a robot arm may be supported on a robot carriage. The robot carriage may bear weight of the robot arm. The robot carriage may support the robot arm. The robot carriage may support a robot arm on a top surface of the robot carriage, a bottom surface of the robot carriage, and/or a side surface of the robot carriage. A robot carriage may support a single robot arm or multiple robot arms. One or more robot arms may be affixed to the carriage or may be movable relative to the robot carriage at the location where the robot is supported by the robot carriage. Robot arms supported by the robot carriage may have the same characteristics or may have one or more differing characteristics (e.g., size, number/type/direction of joints, number/type/characteristics of support members, end effectors, materials, etc.).

The robot carriage may be capable of motion. The robot carriage may move relative to the rest of the workcell. The robot carriage may move relative to one or more bioreactors. The robot carriage may move relative to equipment used for seed train preparation, and/or sample handling. The robot carriage may be supported be a support mount, such as a linear rail 252. The robot carriage may move in a translational manner along the support mount. For instance, the robot carriage may move laterally and/or vertically along a support mount. The support mount may comprise one or more straight lines, curves, and/or corners. The support mount may be formed from a single track or may comprise multiple tracks that the robot carriage may follow. A support mount may be elevated. The support mount may be supported by a workcell floor, wall, and/or ceiling. In some instances, a location of a robot may be measure and/or monitored with aid of the support mount. In some instances the support mount may have a known location and the location of the robot carriage relative to the support mount may be determined. In some embodiments, one or more motors and/or sensors may be provided on a support mount, such as a linear rail, to effect movement of the robot carriage. Optionally, one or more motors and/or sensors may be provided on a robot carriage to effect movement of the robot carriage.

In some instances, the robot carriage may be capable of movement without being restricted to a track or rail. The robot carriage may move autonomously or semi-autonomously. In some instances, the robot carriage may move across a surface. For example, one or more sets of wheels, legs, arms, treads, gliders, or other components may be used to propel a robot carriage. A robot carriage may drive along a floor of a workcell. A robot may be supported by a quadcopter or other type of flying vehicle. A robot may be capable of flight within a workcell.

The robot carriage may optionally bear weight of one or more bulk media containers 240. The robot carriage may or may not support one or more bulk media containers. One or more bulk media containers may move with the robot carriage. For instance, if a robot carriage navigates a rail, the bulk media containers may move along with the robot carriage along the rail.

A location and/or position of the robot may be monitored. In some embodiments, one or more sensors on a robot arm, robot carriage, support mount, or other portion of the workcell may be used to determine the location of the robot within the workcell and position of one or more components of the robot. This may be useful when the robot needs to execute precise motions in interacting with various components of the workcell. In one examples, servomotors may be employed that may be useful for determining position, speed, or acceleration of the robot, or one or more components of the robot.

In one example, a robot may comprise an end effector. For instance, one or more end effectors may be positioned at an end of a robot arm. In some instances, end effectors may be provided at other locations along a robot arm. An end effector may interact with one or more other component of a workcell. For instance, an end effector may manipulate or interact with one or more containers or equipment. An end effector may be used to lift and/or transport a container. An end effector may be used to rotate or flip a container. An end effector may be used to interact with equipment (e.g., press a button, flip a switch, turn a dial, open/close a door, touch a touchscreen, etc.).

Various types of end effectors may be employed. In one example, an end effector may comprise a gripper. A gripper may grasp one or more objects. A gripper may comprise two or more ‘fingers’ that may be capable of movement relative to one another. A gripper may be moved relative to the rest of the arm and allow an object held by the gripper to move rotationally and/or translationally.

In some embodiments, an end effector may utilize magnets, vacuum suction, fasteners, cutters, sensors (e.g., cameras, barcode readers, microphones, etc.), emitters (e.g., light, sound), or other components to sense and/or interact with other components of the workcell. In one example, an end effector may comprise a pipettor. In another example, an end effector may comprise an optical detector, such as a camera or barcode reader. Different types of end effectors may be provided. In some instances, multiple of the same type of end effectors may be provided. They may have the same dimensions or other characteristics, or different dimensions or other characteristics.

An end effector may move in any direction. For instance, an end effector supported by a robotic arm, may translate along one or more, two or more, or three or more axes, or may rotate about one or more, two or more, or three or more axes. An end effector may rotate about a roll axis, pitch axis, and/or yaw axis.

In some embodiments, multiple types of end effectors may be utilized by a robot. The end effectors may be swappable 253. For example, a first end effector may be removed from a robot arm. A second end effector may then be attached from the robot arm. The first end effector and the second end effector may be of the same type or different types. The first end effector and the second end effectors may have the same characteristics or may have at least one characteristic that is different. In some instances, a robot arm may utilize a single end effector at a time. Alternatively, a robot arm may be capable of utilizing multiple end effectors at a time. A workcell may have one or more locations where end effectors that are not being used by the robot are stored. The robot may drop off and/or pick up new end effectors as needed. The robot may swap end effectors according to need. In some embodiments, a workcell may comprise multiple robots. The multiple robots may share the same pool of end effectors. Alternatively, each robot may have its own set of end effectors that it may access.

Any number of robots may be selected for a workcell. In some embodiments, a single robot may be provided for a workcell. In other instances, multiple robots may be selected for a workcell. In some embodiments, the number of robots may depend on a number of bioreactors at a fermentation station. For example, at least one robot may be provided for each at least 1 bioreactor, 2 bioreactors, 3 bioreactors, 4 bioreactors, 6 bioreactors, 8 bioreactors, 10 bioreactors, 12 bioreactors, 18 bioreactors, 24 bioreactors, 36 bioreactors, 48 bioreactors, 60 bioreactors, or 96 bioreactors. In some instances, selected robots may have selected roles and not perform other roles that performed by other robots. In some instances, each robot may be capable of performing any role within the workcell. In some embodiments, a selected robot may only interact with selected bioreactors while not interacting with other bioreactors. For instance, if two robots are provided, a first robot may interact with bioreactors 1-12 while a second robot may interact with bioreactors 13-24. Alternatively, any of the robots in the workcell may interact with any of the bioreactors on an as-needed basis. For instance, if two robots are provided, both the first and second robots may be capable of interacting with any of bioreactors 1-24. In another example, selected robots may only interact with certain stations or sets of equipment. For instance, a selected robot may interact with a seed train station while not interacting with the sample handling station. Alternatively, any of the robots may interact with any of the stations or any of the equipment.

A work cell may comprise one or more sensors for environmental monitoring 260. The environmental monitoring sensors may be capable of detecting one or more conditions within a workcell. The sensors may detect conditions at particular regions or stations of the workcell, or the workcell overall. The sensors may include, and are not limited to, temperature sensors, pressure sensors (e.g., air pressure sensor), gas detectors (e.g., detecting ambient O₂, CO₂, or other gases), motion sensors, particulate sensors, microphones, optical sensors, or other sensors. The sensors may be useful for detecting whether the environment is sterile or whether contamination has occurred. The sensors may be useful for detecting an error condition. The sensors may be useful for detecting whether the environment is conducive to various experimental parameters for the bioreactors.

A work cell may comprise one or more cameras 270. A camera may monitor activity within the workcell. In some instances, data collected by a camera may be automatically analyzed with aid of one or more processors. In some instances, a human may view images captured by a camera in real-time or at a later time. In some instances, cameras may be useful for monitoring completion and timing of tasks. This may be useful for determining when human tasks are required, for example, when doors open. For instance, cameras may be useful for determining when experiments are complete.

A single camera may be provided within the workcell. Alternatively, multiple cameras may be provided within a workcell. Different cameras may have different fields of view. For instance, different cameras may be used to capture images of different regions of the workcell. The cameras may have a fixed position relative to the rest of the workcell. Alternatively, the cameras may be movable relative to the rest of the workcell. In some examples, the cameras may rotate about one, two, three or more axes. The motion of the camera may optionally be remotely controlled from outside the workcell.

The cameras may be useful for detecting whether a contamination or accident has occurred. The cameras may be useful for detecting an error condition.

A workcell may optionally comprise one or more emitters. For example, one or more light sources may be provided within the workcell. An emitter may emit visible light, UV light, IR radiation, sound, or provide any other form of emission.

A workcell may or may not be ventilated. A controlled ventilation system may or may not be provided for a workcell.

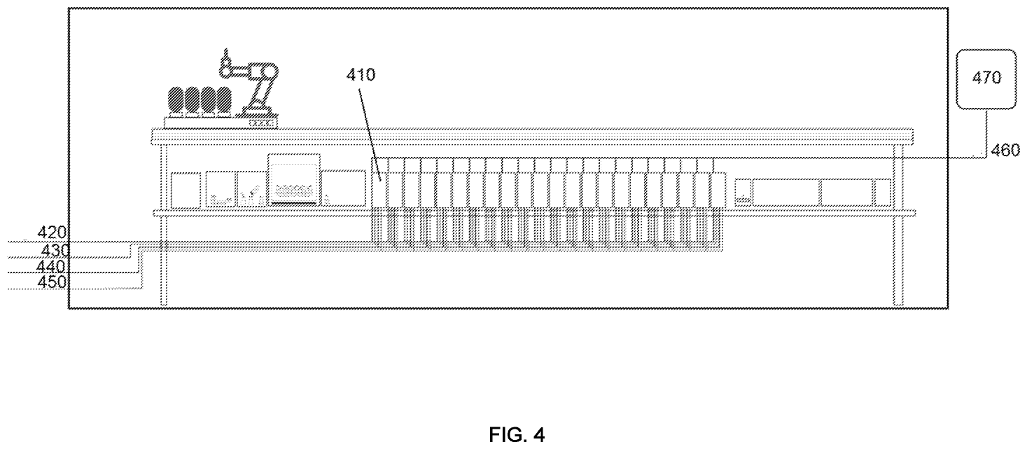

FIG. 4 shows examples of manifolds for bioreactors, in accordance with embodiments of the invention. The manifolds may comprise gas, liquid, electricity, and/or data manifolds. In some embodiments, each bioreactor, when plugged in within a workcell, may be coupled to the various manifolds.

A workcell may comprise one or more bioreactors 410. Each bioreactor may be removable from a workcell and/or re-attachable. The bioreactors may be swapped in and out as desired. The bioreactors can be swapped, for example, mid-run (e.g., on-the-fly or hot-swapped). Each bioreactor may be capable of operating independently of another bioreactor. A bioreactor may be received at a receiving interface of the workcell. When the bioreactor is attached to the receiving interface, the bioreactor may be capable of operation. If the bioreactor is not attached to a receiving interface, the bioreactor may be inoperable.

A bioreactor may receive inlet gas 420, electricity 430, data 440, and/or liquid 450. In some embodiments, outlet gas 460 may be removed from a bioreactor. When a bioreactor is attached to a receiving interface of a workcell, the bioreactor may be coupled to various types of input/output (I/O) interfaces, such as the various inlet gas, electricity, data, and/or liquid interfaces. The various I/O interfaces may allow one or more components of the bioreactor to receive one or more inputs and/or provide one or more outputs (e.g., of power, data, gas, or liquid).

In some embodiments, one or more I/O interfaces may be coupled to a manifold. The manifold may branch off into multiple paths for the individual I/O interfaces. In some embodiments, a single manifold may be provided which may branch off into the individual I/O paths at the same level. Alternatively, any number or level of branches may be provided for the various paths. An I/O interface may couple to a single bioreactor. In some instances, the input or output at each interface may be individually controlled. The input or output each interface may operate independently of other interfaces. For example, the gas flowing into a first bioreactor may be controlled independently of gas flowing into a second bioreactor. In some instances, the manifold may be provided as a bus. For example, electricity and/or data may be provided via a bus.

A bioreactor may receive inlet gas 420 via an inlet gas interface. Any type of inlet gas may be provided. In some embodiments, the inlet gas may be 02, CO2, air, and/or nitrogen. The flow of gas may be controlled. In some embodiments, the flow of gas may be turned on or off. The rate of gas flow may be controlled (e.g., maintained, increased, or decreased). In some instances, multiple types of gases may be provided. Each type of gas may have its own input interface. Each type of gas may be individually and/or independently controllable. In some instances a gas inlet may comprise a mixture of gases. The flow of gas may be controlled locally from within the workcell or from outside the workcell. For example, a user at a remote location may control the flow of gas. One or more processors may automatically determine a desired flow of gas, optionally without requiring human intervention. In some embodiments, the inlet gas may enter one or more components of the bioreactor. In one example, the inlet gas may enter a reactor vessel. The inlet gas may aid in a fermentation process. In some instances, a mass flow controller may control the flow rate of the inlet gas. The mass flow controllers may be provided within each bioreactor.

The source of the inlet gas may be provided within the workcell. Alternatively, the source of the inlet gas may be provided from outside the workcell. The flow of gas may be controlled with aid of one or more pumps.

A bioreactor may receive electricity 430 via a power interface. A bioreactor may or may not have a local power source. In some embodiments, a bioreactor may receive power from a power source external to the bioreactor. The power source may be within a workcell. For instance, an energy storage and/or generation system may be provided within a workcell. In some embodiments, a workcell may receive power from outside a workcell. For instance, the workcell may receive power from a utility grid. The electricity provided to the bioreactor may be from outside the workcell, such as a utility grid. In some instances, an entirety of the bioreactor's power may come via the power interface. The bioreactor may be incapable of any operation when detached from the rest of the receiving interface of the workcell. Alternatively, a portion or an entirety of the bioreactor's power may come from a power source on-board the bioreactor. The bioreactor may optionally be capable of some limited operations or regular operation when detached from the rest of the receiving interface of the workcell. In some embodiments, when the bioreactor is attached to the workcell, the electricity may automatically start being provided to the bioreactor. This may optionally start an initialization process. Data may be exchanged between the bioreactor and another portion of the workcell or outside the workcell via the initialization process.

A data communications 430 may occur between a bioreactor and one or more external devices via a data interface. In some embodiments, data communications may be provided to a bioreactor. In some instances, such data communications may include instructions that may pertain to operation of the bioreactor. In some instances, the bioreactor may comprise one or more local controllers that may provide instructions that may affect operation of the bioreactor. The local controllers may receive instructions via data communications that may or may not affect the instructions provided by the local controllers. In some instances, local controllers may not be needed and instructions received by the data communications may directly control operations of the bioreactor.

Data communications may be provided from a bioreactor. In one example, data collected by one or more sensors may be provided from a bioreactor. The data may optionally be provided to one or more external device, such as a computer. Possible communication architectures are provided in greater detail elsewhere herein. In some instances, data communications from the bioreactor may comprise status information about the bioreactor. For example, the data communications may indicate whether the bioreactor is operational, whether the bioreactor is off, whether there is an error state associated with the bioreactor, or any other information. The data communications from the bioreactor may include information about a bioreactor's current stage of an experiment.

Two way communications may be provided via a data interface. In some embodiments, a data interface may detect when a bioreactor is attached to a receiving interface or when a bioreactor is detached from a receiving interface. In some instances, when a bioreactor is attached to a receiving interface an initialization process may occur. For example, the bioreactor may receive power from the power interface and automatically provide information about the bioreactor (e.g., bioreactor identity, bioreactor configurations and modules, bioreactor status, etc.). This may allow for a modular workcell system, where various bioreactors may operate independently of one another and may optionally have different configurations.

A bioreactor may receive water 450, or another liquid, via a liquid interface. Any type of liquid may be provided. In some embodiments, the liquid may be water. The water may be a cold water or hot water. In some instances, the liquid may be a coolant. Any coolant known or later developed in the art may be provided, such as propylene glycol. The flow of liquid may be controlled. In some embodiments, the flow of liquid may be turned on or off. The rate of liquid flow may be controlled (e.g., maintained, increased, or decreased). The temperature of the liquid may be controlled (e.g., maintained, increased, or decreased). In some instances, multiple types of liquid may be provided. Each type of liquid may have its own input interface. Each type of liquid may be individually and/or independently controllable. In some instances a liquid input may comprise a mixture of liquids. The flow of liquid may be controlled locally from within the workcell or from outside the workcell. For example, a user at a remote location may control the flow of liquid and/or a desired liquid temperature. One or more processors may automatically determine a desired flow of liquid, optionally without requiring human intervention. In some embodiments, the liquid may enter one or more components of the bioreactor. In one example, the liquid may enter a reactor vessel. In another example, the liquid may enter a water bath that may be used to heat or cool a reactor vessel. The liquid may aid in a fermentation process. The liquid may be provided via a channel, pipe, tubing, or other mechanism.

The source of the liquid may be provided within the workcell. Alternatively, the source of the liquid may be provided from outside the workcell. The flow of liquid may be controlled with aid of one or more pumps. In some instances, one, two or more liquid feed lines may be provided, which may be controlled by one or more pumps, such as peristaltic pumps.

A gas outlet 460 may be provided. Gas from one or more bioreactors may be conveyed outside the various bioreactors. The gas may be conveyed within or outside the workcell. In some embodiments, the gas may come from one or more components of the bioreactor. In one example, the gas may come from a reactor vessel. The gas may be a byproduct of a fermentation process. The gas may be conveyed via a channel, pipe, tubing, or other mechanism. In some instances, fans or pumps may aid in the flow of the outlet gas.

Optionally, a condenser may be provided on a gas outlet line. The condenser may prevent undesired liquid loss from the materials within a reactor vessel. For example, the condenser may prevent undesired water loss from a fermentation broth.

The outlet gas may optionally be conveyed to an off-gas analysis system 470. The off-gas analysis system may be provided within a workcell or outside a workcell. The off-gas analysis system may optionally automatically analyze the outlet gas without requiring human intervention. The off-gas analysis system may analyze the outlet gas. In some embodiments, the system may analyze outlet gas composition, quantity, density, temperature, or other characteristics.

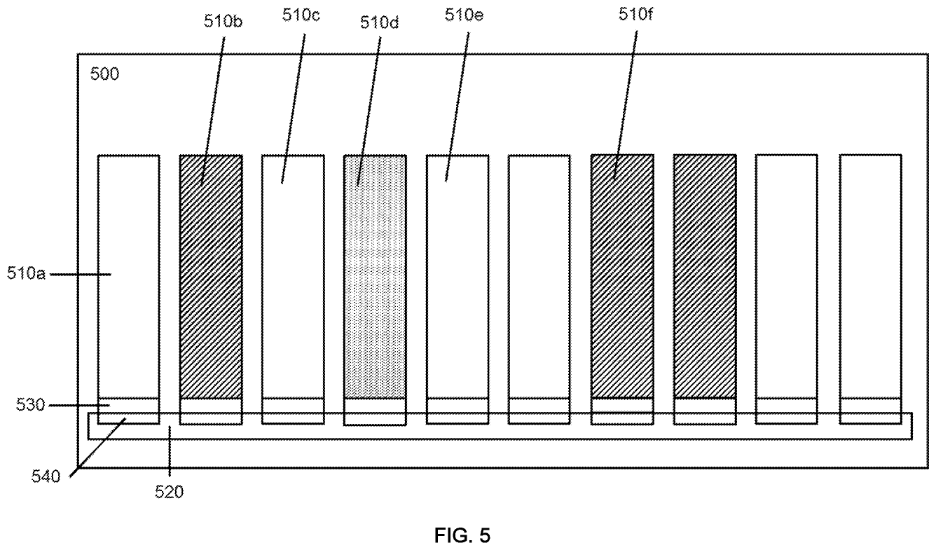

FIG. 5 shows an example of a modular bioreactor array, in accordance with embodiments of the invention. As previously described, a workcell may comprise a bioreactor array 500 which may have any number or arrangement of bioreactors 510a-f.

The bioreactors may be attachable and/or detachable from a portion 520 of the workcell. In some instances, the portion of the workcell may be a substrate or support to which the bioreactors may be attached and/or detached. The portion of the workcell may rest on a workcell floor, or may be elevated with aid of a structure. The portion of the workcell may be integrated into the floor or may be the floor itself, or may be integrated into a structure or may be a structure itself. The portion of the workcell may be formed of a single piece or multiple pieces. Multiple pieces may be continuously connected or may be detached from one another.

A bioreactor may couple to one or more receiving interfaces 540 of a workcell. In some embodiments, a single bioreactor may couple to a single receiving interface. In some instances, any number or arrangement of receiving interfaces may be provided. Any portion of the receiving interfaces may have a bioreactor attached thereon. In some instances, experiments may be run on bioreactors that are attached, without requiring that all receiving interfaces have a bioreactor attached thereon. The experiments on the bioreactors attached to the receiving interfaces may be run in parallel. The experiments on the bioreactors attached to the receiving interfaces may be run independently of one another. The may have the same or may have different start times and/or end times. An experiment may automatically begin when a bioreactor is attached to a receiving interface. In another example, an experiment may begin in response to an instruction to begin, which may be generated from within the workcell, or outside the workcell.

A mechanical connection may be provided between the bioreactor and the receiving interface. The mechanical connection may permit the bioreactor to couple to the receiving interface in a secured manner (e.g., not allowing the bioreactor to topple over or fall off). Locking mechanisms may optionally be provided to secure the bioreactor to the receiving interface. In some instances, a frictional fit may be provided to secure the bioreactor to the receiving interface. Optionally, one or more fasteners, quick-release mechanisms, magnets, lock-and-slide mechanisms, or other mechanisms may be employed to secure the bioreactor to the receiving interface. A bioreactor may have a corresponding connection interface 530 that may couple to the receiving interface. In some instances, the corresponding connection interface may have a shape that may be complementary to the receiving interface. The connection interface may allow for a mechanical connection between the bioreactor and the receiving interface.

One or more power and/or data communications may occur via the connection interface and the receiving interface. Electrical connection may be provided between the bioreactor and the portion of the workcell. Power and/or data may flow to or from the bioreactor via the interfaces. In some instances, electrical contacts may be provided between the connection interface and the receiving interface. Communication ports may be utilized to convey data. For example, serial ports, USB ports, Firewire ports, or any other ports known or later developed in the art may be utilized for communication. Contact between the bioreactor and the receiving interface may permit the power and/or data flow. In some instances, the data and/or power flow may occur after the bioreactor is mechanically secured to the receiving interface. A connection interface may have complementary communication and power portions that may couple to and/or contact the corresponding communication and power portions of the receiving interface.

One or more gas and/or liquid flows may occur via the connection interface and the receiving interface. Liquid and/or gas may flow to or from the bioreactor via the interfaces. In some instances, connection portions for different flow portions may be provided between the connection interface and the receiving interface. Contact between the bioreactor and the receiving interface may permit the liquid and/or gas flow. In some instances, the liquid and/or gas flow may occur after the bioreactor is mechanically secured to the receiving interface. A connection interface may have complementary gas and/or liquid portions that may couple to and/or contact the corresponding gas and/or liquid portions of the receiving interface. A fluid-tight seal may be formed when the various gas and/or liquid portions come into contact with one another. A fluid-tight seal may be formed when the bioreactor is mechanically secured to the receiving interface. In some instances, gas and/or liquid flow may not start until the sealing is confirmed. The sealing may prevent leakage of the gas and/or liquid. In some instances, one or more sensors may be provided to confirm whether sealing has occurred and is adequate. In some instances, liquid and/or gas flow may be automatically stopped when it is detected that leakage has occurred or that the seal is no longer sufficient. The liquid and/or gas flow may be automatically stopped during an experiment. In some instances, an alert may be provided to a user when leakage has occurred or seal is no longer sufficient.

In some embodiments, the receiving interfaces within a workcell may have identical configurations. In some instances, the connection interfaces of the bioreactors may have identical configurations, regardless of other differences that may exist for the bioreactors. This may allow for any bioreactor to be attached to any receiving interface within the workcell, regardless of the bioreactor configuration. This may allow for universal swapping in and out of bioreactors. In other instances, different receiving interfaces within a workcell may have different configurations. In some instances, only certain subsets of bioreactors may fit with certain subsets of receiving interfaces.

The various bioreactors may advantageously allow for modular operation within the workcell. The bioreactors may be swapped in for one another. The swapping can happen without shutting down the system, and can happen during the middle of a fermentation run. Bioreactors may be used for various experiments. In some instances, the instructions for the various experiments may be provided locally at the bioreactors. Different experiments may have different sets of instructions. Bioreactors with different sets of instructions may be swapped in and out for one another. One or more controllers of the bioreactors may have various sets of instructions that may be preprogrammed or fixed, to accommodate different experiments. The bioreactors with different sets of instructions may or may not have any physical differences.

In some instances, the various bioreactors utilized for different experiments may have one or more different physical characteristics. For example, a first bioreactor 510a may be used for a different experiment than a second bioreactor 510b. The first bioreactor may have one or more different physical characteristics to accommodate the different experiments. For example, the first bioreactor may have different temperature control system configurations than the second bioreactor. In another example, a first bioreactor may accommodate a different reactor vessel size and/or shape compared to the second bioreactor. The first bioreactor may utilize different sensors than the second bioreactor. The first bioreactor may accommodate different gas and/or liquid flows relative to the second bioreactors.

In some embodiments, within a workcell, two or more of the bioreactors (e.g., 510a, 510c, 510e) may have the same configurations. The bioreactors having the same configurations may optionally be used to run the same experiment or run different experiments. Within a workcell, two or more bioreactors may have different configurations (e.g., 510a and 510b, or 510d and 510e). In some instances, a bioreactor within a workcell may have a unique configuration 510d that is not shared by any of the other bioreactors in the workcell. Any number of bioreactors, and/or number of configurations of bioreactors may be used in a workcell at a given time.

In some instances, bioreactors with different built-in configurations may be swapped in and out for one another. Optionally, the bioreactors themselves may be modified. Different components of the bioreactors may be swapped in and out for each other. Then the reconfigured bioreactors may be attached to the workcell.

Any description herein of modularity for bioreactors may apply to any type of equipment within the workcell. For instance, different equipment utilized for seed train preparation and/or sample handling may be swapped in and out for one another. Different analytical equipment and/or sample preparation equipment may be exchanged or modified. For example, a first type of centrifuge may be exchanged for a second type of centrifuge that has different physical characteristics. In another example, a mass spectrometer may be exchanged for a gas chromatography device.

The various types of equipment may or may not be received by a receiving interface. In some embodiments, the various types of equipment may or may not have designated regions within the workcell where they are placed. In some instances, one or more sensors may be utilized to detect the various equipment provided within the workcell. For example, cameras or barcode scanners may be used to detect the presence of selected equipment, and/or determine the location of the selected equipment within the workcell. In some instances, a human operator may be able to swap the various equipment in and out without providing any additional inputs. Alternatively, a human operator may indicate when equipment has been swapped in or out. One or more processors may be utilized to automatically recognize and/or detect the equipment that is within the workcell. In one example, an image collected by a camera may be analyzed with aid of one or more object or character recognition algorithms to detect the equipment. The information identity and placement of the equipment may be utilized during running of the experiments. The robot may be able to accommodate the various equipment provided when the identity and/or placement of the equipment is known.

The automatic detection and/or identification of the bioreactors and other equipment advantageously permit the system to run the various experiments without requiring a large amount of input or set-up by human operators. This allows for more rapid set-up and running of different experiments, which saves time and cost. This also reduces the likelihood of human error.

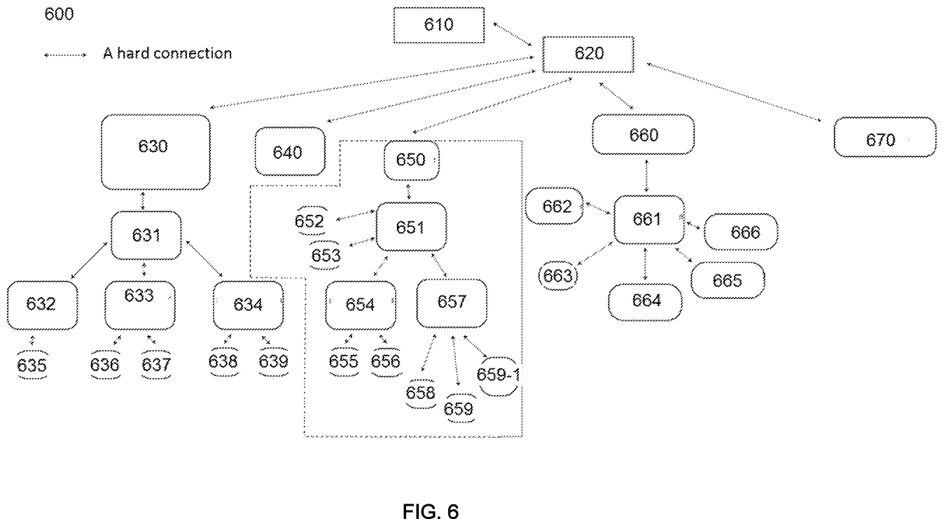

FIG. 6 shows an example of an electronics/communications architecture 600, in accordance with embodiments of the invention. Such architecture is provided by way of example only, and is not limiting. Alternative components or modules may be utilized.

A cloud server 610 may communicate over a network 620 with a workcell. Any description herein of a cloud server may apply to one or more server computers. In some instances, cloud computing infrastructure may be utilized. Optionally peer-to-peer infrastructure may be utilized. A cloud server may be remote to a workcell. The cloud server may optionally be at a different room, building, address, city, state, or country from the workcell, or may not be required to be at any of these locations. The cloud server may be in the same room, building, address, city, state, or country as the workcell.

The cloud server may include or communicate with a remote terminal through which a user may interact with the system. A user may optionally be an individual running one or more experiments in the workcell, or managing the workcell. Optionally, multiple remote terminals and/or users may have access to the system. A remote terminal may optionally be outside a workcell. A remote terminal may be at a different room, building, address, city, state, or country from the workcell.

The cloud server may communicate with the workcell over a network 620. Any type of network may be employed. For instance, a local area network (LAN), or wide area network (WAN), such as the Internet may be employed. The network may be a telecommunications network.

The workcell may comprise a workcell computer 630. The workcell may be physically within the workcell, or may be outside but directly communicating with components of the workcell. The workcell computer may communicate with a workcell embedded system 631. A workcell embedded system may communicate with an environmental sensor embedded system 632 (which may optionally comprise one or more sensors 635, such as sensors used in environmental monitoring 260), a carriage pumping/scales embedded system 633 (which may optionally comprise scales 636, such as scales 212, 231, and/or pumps 637), and/or a linear rail embedded system 634 (which may comprise one or more motors 638 and/or sensors 639, of a linear rail 252).

The workcell computer may or may not have user interface that may allow a user to directly interact with the workcell computer. Communications between the workcell computer and the various systems may be hardwired, or may be wireless. The workcell computer may communicate with the cloud server over a network.

The workcell may comprise a robot 640. The robot may have any characteristics or features as described elsewhere herein. The robot may communicate with a cloud server over a network. The robot may communicate information about a robot status, robot location, robot position, or robot activity. In some instances, a robot may receive instructions from a cloud computer, workcell computer, or any other device as described elsewhere herein. For example, a robot may be instructed to perform a particular task. A robot may be capable of performing steps autonomously or semi-autonomously. In some instances, a robot may be provided with a task, and the robot may be able to execute the task within the environment without detailed direction and based on information collected by sensors (e.g., on-board and/or off-board the robot). A robot may have one or more processors on-board the robot that may be capable of generating instructions for execution by the robot. The robot may have one or more processors that may execute code, logic or instructions for performing one or more steps. The robot may have one or more memory storage unit that may comprise non-transitory computer readable media comprising code, logic, or instructions for performing one or more steps.

In some embodiments, a bay computer 650 may be provided in a workcell. A bay computer may be provided for each bay (e.g., bioreactor). The bay computer may communicate with a bay embedded system 651, which may communicate with one or more sensors 652 and/or motors 653 for the bay. The bay embedded system may optionally communicate with a pumping embedded system 654, which may comprise one or more pumps 655 and/or scales 656. The bay embedded system may communicate with a thermal embedded system 657 which may comprise sensors 658, pumps 659, and/or heaters/coolers 659-1. Such systems may be provided for each bay. Such systems may be capable of operating independently of systems in other bays.

The bay computer may or may not have user interface that may allow a user to directly interact with the bay computer. Communications between the bay computer and the various systems may be hardwired, or may be wireless. The bay computer may communicate with the cloud server over a network. The bay computer may optionally communicate with a workcell computer, and/or a robot directly or over a network.

A workcell may comprise a seed train computer 660. The seed train computer may interact with a seed train embedded system 661. The seed train embedded system may comprise a bar code reader 662, scales 663, incubator 664, incubator environmental sensors 665, and/or live optical density sensing 666. Any other sensors or equipment described elsewhere herein, including those related to the seed train station, may be included herein.

The seed train computer may or may not have user interface that may allow a user to directly interact with the seed train computer. Communications between the seed train computer and the various systems may be hardwired, or may be wireless. The seed train computer may communicate with the cloud server over a network. The seed train computer may optionally communicate with a workcell computer, a robot, and/or a bay computer directly or over a network.

A sample analysis computer 670 may be provided, optionally within a workcell. The sample analysis computer may optionally interface with any systems or equipment within the workcell. The sample analysis computer may interface with any sample handling sensors, equipment or other components described elsewhere herein.

The sample analysis computer may or may not have user interface that may allow a user to directly interact with the sample analysis computer. Communications between the sample analysis computer and the various systems may be hardwired, or may be wireless. The sample analysis computer may communicate with the cloud server over a network. The sample analysis computer may optionally communicate with a workcell computer, a robot, a bay computer, and/or a seed train computer directly or over a network.

The systems and methods provided herein may allow for an automated fermentation workcell to perform experiments in a fully automated fashion. This may allow for communication between the various components to perform the fermentation processes, which may include the seed train preparation, the fermentation, sample preparation, and/or sample analysis. The systems and methods provided herein also allow the workcell to be modular, which may permit flexibility in running various experiments.

Fermentation

Fermentation processes can be used for many applications. For instance, fermentation can be utilized for production of biomass (e.g., viable cellular material, which can include, for example, phage), production of extracellular metabolites (chemical compounds), production of intracellular components (e.g., enzymes and other proteins), transformation of a substrate (e.g., the substrate itself may be a product), or development of a consortium of bacteria.

A consortium of bacteria can refer to one or more microbial groups living symbiotically. The consortiums can be endosymbiotic or ectosymbiotic. Fermentation processes are useful for biological experiments, drug manufacturing, food industry, biofuels, or many other applications. In some instances, it may be desirable to provide automated fermentation systems and methods that allow for low risk of contamination, high levels of accuracy and repeatability, high throughput, controlled variations, quicker turnaround, and/or require less manpower.

Fermentation can employ a fermentation agent to transform a substrate, for example, carbon, oxygen, nitrogen, hydrogen, sulfur, calcium, and trace elements, into a useful product. Fermentation can be an aerobic process, a process that uses oxygen, or an anaerobic process, a process that can occur in oxygen-limited or oxygen-restricted environments. A fermentation agent can be, for example, yeast, bacteria, algae, fungi, mammalian cells, animal cells, a phage, a bacteriophage, and insect cells, or any combination thereof. The mammalian cells may include, for example, Chinese Hamster Ovaries (CHO), Human Embryonic Kidney (HEK), cell lines mouse myeloma (NSO), newborn hamster kidney (BHK), human embryonic kidney (HEK-293), human retinal cells or cell lines.

Non-limiting examples of a yeast that can be used in a system described herein include Saccharomyces cerevisiae, S. oviformis, S. chevaliers Schizosaccharomyces pombe, S. uvarum, S. carlsbergensis, Sch. malidevorans, Kluyveromyces, Debaromyces, Hanseniaspora, Issatchenkia, Pichia, Candida, S. bisporus, S. rouxii, Zygosaccharomyces; S. roses, Torulaspora; and T. delbrueckii.

Non-limiting examples of a bacterium that can be used in a system described herein include Streptococcus, Lactobacillus, Bacillus, Lactococcus, Lactococcus, Propionibacterium, Escherichia, Enterobacter, Clostridium, Streptococcus lactis, S. cremoris, Leuconostoc mesenteroides, L. dextranicum, L. lactis, L. cremoris, L. paramesenteroides, L. oenos, Pediococcus cerevisiae, P. acidilactici, P. pentosaceus, P. halophilus, Enterococcus, Lacotbacillus, delbrueckii, L. leichmannii, L. lactis, L. bulgaricus, L. helviticus, L. acidophilus, L. casei, L. plantarum, Leuconostoc, Lacotbacillus fermentarum, L. brevis, L. buchneri; may produce manitol and dextran; Lactobacillus, Pediococcus, Leuconostoc, Clostridium butyricum (1, 2); Cl. acetobytylicum (1, 3, 4, 5); Cl. kluyveri, Cl. aceticum (1, 6); Propionigenium modestum, Oxalobacterformigenes; Anaeroplasma and Arthrobacte, and Acetobacterium.

Non-limiting examples of a fungus that can be used in a system described herein include Mucor (e.g., M. miehei), Rhizopus (e.g., R. oligosporus), Alternaria, Aspergillus, A. rugulosus, Botrytis (e.g., B. cinerea), Cladosporium, Colletotrichum, Fusarium, Monilia, Penicillium, Trichothecium, Aureobasidium (Pullularia), Geotrichum, and G. candidum.

The result of fermentation can be, for example, acetic acid, citric acid, ethanol, antibiotics, proteins, enzymes, monoclonal antibodies, the organisms themselves, cheese, wine, or beer.

A fermentable substrate (e.g., a carbon source) can comprise, for example, a polysaccharide, a monosaccharide, a carbohydrate, a sugar, a starch, methane, carbon dioxide, carbon monoxide, or cellulose. In some embodiments, culture media includes complex fermentable substrates containing a variety of carbon sources, such as yeast extract. Media also can include salts, nitrogen sources, trace metal elements, and other components for growing biomass and producing fermentation products.

While various embodiments of the invention have been shown, and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EMBODIMENTS

The following non-limiting embodiments provide illustrative examples of the invention, but do not limit the scope of the invention.

### Embodiment 1

An apparatus for automated fermentation, said apparatus comprising: an automated seed train preparation station; a plurality of modular bioreactors configured to receive seed from the automated seed train preparation station, wherein at least one of the modular bioreactors of said plurality is removable and capable of having a different configuration from at least one other bioreactor of said plurality; and at least one robotic arm configured to aid in automated seed train preparation at the automated seed train preparation station.

### Embodiment 2

The apparatus of embodiment 1, further comprising an automated sample handling station configured to receive a sample from the plurality of bioreactors.

### Embodiment 3

The apparatus of any one of embodiments 1-2, wherein the removal of the at least one of the modular bioreactors of said plurality of bioreactors occurs during a fermentation run.

### Embodiment 4

The apparatus of any one of embodiments 1-3, wherein the at least one of modular bioreactors of said plurality of bioreactors comprises a media bottle.

### Embodiment 5

The apparatus of any one of embodiments 1-4, wherein the at least one of modular bioreactors of said plurality of bioreactors comprises a single use reactor.

### Embodiment 6

The apparatus of any one of embodiments 1-5, wherein the at least one of modular bioreactors of said plurality of bioreactors comprises a magnetic agitation system.

### Embodiment 7

The apparatus of any one of embodiments 1-6, wherein the at least one of modular bioreactors of said plurality of bioreactors comprises a heater.

### Embodiment 8

The apparatus of any one of embodiments 1-7, wherein the at least one of modular bioreactors of said plurality of bioreactors comprises a cooler.

### Embodiment 9

The apparatus of any one of embodiments 1-8, further comprising a sensor, wherein the sensor is contained within the at least one of the modular bioreactors of said plurality of bioreactors.

### Embodiment 10

The apparatus of embodiment 9, wherein the sensor measures an optical density of a culture in the at least one of the modular bioreactors of said plurality of bioreactors.

### Embodiment 11

A system for automated fermentation, said system comprising: an automated seed train preparation station comprising a fermentation agent; a plurality of modular bioreactors configured to receive seed from the automated seed train preparation station, wherein at least one of the modular bioreactors of said plurality is removable and capable of having a different configuration from at least one other bioreactor of said plurality; and at least one robotic arm configured to aid in automated seed train preparation at the automated seed train preparation station.

### Embodiment 12

The system of embodiment 11, further comprising an automated sample handling station configured to receive a sample from the plurality of bioreactors.

### Embodiment 13

The system of any one of embodiments 11-12, further comprising a sterile enclosure around the automated seed train preparation station, the plurality of bioreactors, the at least one robotic arm, and the automated sample handling station, configured to prevent undesirable contaminants from entering the enclosure.

### Embodiment 14

The system of any one of embodiments 11-13, further comprising a camera to monitor activity of the system for fermentation.

### Embodiment 15

The system of any one of embodiments 11-14, wherein said fermentation agent is used to inoculate said plurality of modular bioreactors.

### Embodiment 16

The system of any one of embodiments 11-15, wherein the fermentation agent is yeast.

### Embodiment 17

The system of any one of embodiments 11-15, wherein the fermentation agent is a bacterium.

### Embodiment 18

The system of any one of embodiments 11-15, wherein the fermentation agent is an alga.

### Embodiment 19

The system of any one of embodiments 11-15, wherein the fermentation agent is a fungus.

### Embodiment 20

The system of any one of embodiments 11-15, wherein the fermentation agent is a mammalian cell.

### Embodiment 21

The system of any one of embodiments 11-15, wherein the fermentation agent is an animal cell.

### Embodiment 22

The system of any one of embodiments 11-15, wherein the fermentation agent is an insect cell.

### Embodiment 23

The system of any one of embodiments 11-22, further comprising a sensor, wherein the sensor is contained within the at least one of the modular bioreactors of said plurality of bioreactors.

### Embodiment 24

The system of embodiment 23, wherein the sensor measures an optical density of a culture in the at least one of the modular bioreactors of said plurality of bioreactors.

### Embodiment 25

The system of any one of embodiments 11-24, further comprising a sensor, wherein the sensor is contained within the sterile enclosure.

### Embodiment 26

The system of embodiment 25, wherein the sensor measures a temperature within the sterile enclosure.

### Embodiment 27

The system of embodiment 25, wherein the sensor measures an oxygen level within the sterile enclosure.

### Embodiment 28

The system of embodiment 25, wherein the sensor measures a carbon dioxide level within the sterile enclosure.

### Embodiment 29

The system of embodiment 25, wherein the sensor measures a pressure level within the sterile enclosure.

### Embodiment 30

The system of any one of embodiments 11-29, wherein the removal of the at least one of the modular bioreactors of said plurality of bioreactors occurs during a fermentation run.

### Embodiment 31

A method for automated fermentation, said method comprising: a system comprising: an automated seed train preparation station; a plurality of modular bioreactors configured to receive seed from the automated seed train preparation station, wherein at least one of the modular bioreactors of said plurality is removable and capable of having a different configuration from at least one other bioreactor of said plurality; and at least one robotic arm configured to aid in automated seed train preparation at the automated seed train preparation station.

### Embodiment 32

The method of embodiment 31, wherein the system further comprises an automated sample handling station configured to receive a sample from the plurality of bioreactors.

### Embodiment 33

The method of any one of embodiments 31-32, wherein the system further comprises a sterile enclosure around the automated seed train preparation station, the plurality of bioreactors, the at least one robotic arm, and the automated sample handling station, configured to prevent undesirable contaminants from entering the enclosure.

### Embodiment 34

The method of any one of embodiments 31-33, wherein the system further comprises a camera to monitor activity of the system for automated fermentation.

### Embodiment 35

The method of any one of embodiments 31-34, wherein the seed is a fermentation agent.

### Embodiment 36

The method of embodiment 35, wherein the fermentation agent is yeast.

### Embodiment 37

The method of embodiment 35, wherein the fermentation agent is a bacterium.

### Embodiment 38

The method of embodiment 35, wherein the fermentation agent is an alga.

### Embodiment 39

The method of embodiment 35, wherein the fermentation agent is a fungus.

### Embodiment 40

The method of embodiment 35, wherein the fermentation agent is a mammalian cell.

### Embodiment 41

The method of embodiment 35, wherein the fermentation agent is an animal cell.

### Embodiment 42

The method of embodiment 35, wherein the fermentation agent is an insect cell.

### Embodiment 43

The method of any one of embodiments 31-42, wherein the system further comprises a sensor, wherein the sensor is contained within the at least one of the modular bioreactors of said plurality of bioreactors.

### Embodiment 44

The method of embodiment 43, wherein the sensor measures an optical density of a culture in the at least one of the modular bioreactors of said plurality of bioreactors.

### Embodiment 45

The method of any one of embodiments 31-44, wherein the sterile enclosures contains a sensor.

### Embodiment 46

The method of embodiment 45, wherein the sensor measures a temperature within the sterile enclosure.

### Embodiment 47

The method of embodiment 45, wherein the sensor measures an oxygen level within the sterile enclosure.

### Embodiment 48

The method of embodiment 45, wherein the sensor measures a carbon dioxide level within the sterile enclosure.

### Embodiment 49

The method of embodiment 45, wherein the sensor measures a pressure level within the sterile enclosure.

### Embodiment 50

The method of any one of embodiments 31-49, wherein the removal of the at least one of the modular bioreactors of said plurality of bioreactors occurs during a fermentation run.

### Embodiment 51

The method of any one of embodiments 31-50, wherein the automated fermentation is for production of biomass.

### Embodiment 52

The method of any one of embodiments 31-50, wherein the automated fermentation is for production chemical compounds.

### Embodiment 53

The method of any one of embodiments 31-50, wherein the automated fermentation is for production of an enzyme.

### Embodiment 54

The method of any one of embodiments 31-50, wherein the automated fermentation is for production of a protein.

### Embodiment 55

The method of any one of embodiments 31-50, wherein the automated fermentation is for production of a phage.

### Embodiment 56

The method of embodiment 43, wherein the sensor measures a liquid level of a culture in the at least one of the modular bioreactors of said plurality of bioreactors.

57. The method of embodiment 43, wherein the sensor measures a foam level of a culture in the at least one of the modular bioreactors of said plurality of bioreactors.

### Embodiment 58

The method of embodiment 43, wherein the sensor measures emitted infrared light of a culture in the at least one of the modular bioreactors of said plurality of bioreactors.

### Embodiment 59

The method of embodiment 43, wherein the sensor measures emitted ultraviolet light of a culture in the at least one of the modular bioreactors of said plurality of bioreactors.

### Embodiment 60

The method of embodiment 43, wherein the sensor measures emitted visible light of a culture in the at least one of the modular bioreactors of said plurality of bioreactors.

What is claimed is:

1. A system for automated fermentation, the system comprising:
an automated seed train preparation station;
a plurality of modular bioreactors configured to receive seed from the automated seed train preparation station; and
one or more processors configured to control the automated seed train preparation station or the plurality of modular bioreactors, and process sensor data to monitor real-time conditions,
wherein the one or more processors are in communication with a cloud server over a network to receive instructions for affecting an operation of the automated seed train preparation station or selected modular bioreactors before or during a fermentation run.

2. The system of claim 1, wherein the sensor data is collected from one or more sensors including a camera to monitor activity of the system for fermentation.

3. The system of claim 2, wherein the camera measures an optical density of a culture in at least one of the modular bioreactors of the plurality of bioreactors.

4. The system of claim 1, wherein the real-time conditions comprise an error condition.

5. The system of claim 2, wherein the camera or the one or more processors are configurable by the cloud server for monitoring the real-time conditions.

6. The system of claim 1, wherein the instructions are provided by a user via a remote terminal.

7. The system of claim 1, further comprising a robotic component configured to aid in automated seed train preparation at the automated seed train preparation station.

8. The system of claim 1, wherein the automated seed train preparation station comprises a fermentation agent.

9. The system of claim 8, wherein the fermentation agent is a mammalian cell.

10. The system of claim 1, wherein at least one of the modular bioreactors of the plurality is removable during the fermentation run.

11. The system of claim 1, wherein the instructions comprise different protocols or processes that can be executed individually by the plurality of modular bioreactors for the same fermentation run.
